(19) United States
(12) Patent Application Publication
(10) Pub. No.: US 2022/0387777 A1
(43) Pub. Date: Dec. 8, 2022

(54) PURGE VALVE ASSEMBLIES SYSTEMS AND METHODS

(71) Applicant: Regents of the University of Minnesota, Minneapolis, MN (US)

(72) Inventors: Jonathan Strutt, Edina, MN (US); Vidhu Pandey, Minneapolis, MN (US)

(73) Assignee: Regents of the University of Minnesota, Minneapolis, MN (US)

(21) Appl. No.: 17/748,809

(22) Filed: May 19, 2022

Related U.S. Application Data
(60) Provisional application No. 63/208,260, filed on Jun. 8, 2021.

(51) Int. Cl. A61M 39/24; A61M 39/26; A61J 1/10

(52) U.S. Cl. CPC A61M 39/24; A61M 39/26; A61J 1/10; A61M 2005/1403

(57) ABSTRACT

Aspects include systems having an intravenous fluid bag, a tubing line assembly including tubing connected to a connector and a purge valve assembly including a spike and an outer housing. The outer housing is connected to the spike and includes a receiving end portion configured to receive the connector. The purge valve assembly further has a check valve provided within the outer housing. Generally, the purge valve assembly interconnects the fluid bag to the fluid tubing assembly for removal of air from the fluid bag prior to connection to the tubing line assembly. Various methods and purge valve assemblies provide a rapid infusion system for use. The purge valve assemblies do not require the user to reinsert the connector or spike into the intravenous fluid bag, creating a sterile solution to preparing the intravenous fluid for rapid infusion while reducing the risk of air emboli.

### RELATED APPLICATIONS

This Non-Provisional patent application claims the benefit of the filing date of U.S. Provisional Patent Application No. 63/208,260, filed Jun. 8, 2021, entitled “PURGE VALVE ASSEMBLIES SYSTEMS AND METHODS,” the entire teachings of which are incorporated herein by reference.

### FIELD OF THE DISCLOSURE

The present disclosure relates to devices and methods of priming intravenous tubing systems including an intravenous (IV) fluid one-way check valve for infusion of IV fluids. Aspects of the disclosure are particularly useful for the rapid infusion of IV fluids.

### BACKGROUND

A rapid infuser device is often used when delivering warmed fluids or blood products stored in a fluid bag to patients in shock requiring immediate resuscitation. Fluid bags used for infusion are prepared with some air in the bag. Various current methods for performing this task involve puncturing the fluid bag with an intravenous tubing spike, and then removing the spike to manually “burp” or extract air from the fluid bag prior to reinsertion of the same tubing spike into the fluid bag.

For most infusion pumps, the same process of “spiking” and “priming” the tubes is employed since flow rates are still low enough for small air bubbles to have minimal physiological effects on the patient. However, the situation differs for rapid infuser systems. Rapid infusion systems are typically used when a patient is in traumatic or hemodynamic emergencies, for they need quick infusions of fluids and/or blood. While some rapid infusion systems include air filtration systems, this alone can sometimes not be enough for larger air bubbles. Therefore, air is manually removed from the intravenous bag through a process known as “burping.” While burping, the fluid bag is flipped over such that the air in the bag is next to the intravenous tubing port. Since fluid/air can only escape from an intravenous fluid bag after it has been spiked, an intravenous tube is used for spiking before being subsequently removed. Then, a health practitioner puts pressure on the intravenous fluid bag to force out the air before putting the intravenous fluid bag right-side up again and reattaching the previously used potentially unsterile intravenous tube.

The present disclosure addresses problems and limitations associated with the related art.

### SUMMARY

Aspects of the disclosure provide a purge valve assembly including an intermediate check valve that does not require the user to reinsert a spike of a fluid tubing assembly or a spike of the purge valve assembly into an intravenous fluid bag, creating a more sterile solution to preparing the intravenous fluid for rapid infusion while reducing the risk of air embolisms. When priming the intravenous tubing line assembly for rapid infusion with a rapid infuser device, any air in the fluid bag is extracted. Typically, the method required to perform this task involves spiking or puncturing the fluid bag with the intravenous tubing line assembly spike provided. The user then must disconnect the spike from the fluid bag, turn the fluid bag over so that the air is near the bag valve, “burp” the bag until all the air has been extracted, and then re-spike the fluid bag with the same intravenous tubing, which is likely no longer sterile and could potentially reintroduce air into the system or fluid bag. In addition to potential contamination, burping the bag risks spilling fluid, making the burping process inefficient and potentially costly. This method involves multiple spikes of the fluid bag with the same device which both increases the risk of contamination with bacteria into the bag and tubing system and also decreases the tightness of the connection at the junction between the fluid bag and the fluid contained therein. Aspects of the disclosure eliminate the need for a second puncture of the fluid bag by creating a method of air extraction that involves insertion of the purge valve assembly between the fluid bag and a tubing line assembly. Systems of the disclosure allows the user to quickly extract air from the fluid bag with the purge valve assembly prior to connecting the tubing line assembly, which can subsequently be attached to the purge valve assembly, completing a setup for rapid infusion.

In one aspect, the present disclosure provides a system including an intravenous fluid bag, a tubing line assembly including tubing connected to a connector and a purge valve assembly. The purge valve assembly includes a spike and an outer housing. The outer housing is connected to the connector and includes a receiving end portion configured to receive the connector. The purge valve assembly further includes a check valve provided within the outer housing.

In one aspect, the present disclosure provides a method including providing an intravenous fluid bag containing fluid and air. In some examples, the fluid bag is made of a flexible material. The method further includes providing a tubing line assembly terminating at a connector providing a purge valve assembly having a spike and an outer and housing maintaining a check valve. The method includes puncturing the fluid bag with the spike and then purging air from the fluid bag, through the spike and out of the check valve. The method can further include connecting the outer housing to the connector.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a partially, exploded side view of a system including a fluid bag, purge valve assembly and tubing line assembly.

FIG. 2 is an exploded side view of the purge valve assembly of FIG. 1.

FIGS. 3A and 3B are cross-sectional views of a check valve of the purge valve assembly of FIG. 2 illustrating the one-way flow restrictive nature of the check valve.

FIG. 4A is a side view of the purge valve assembly of FIGS. 1-2.

FIG. 4B is an isometric view of the purge valve assembly of FIGS. 1-2 and 4A.

FIG. 4C is a reverse isometric view of the purge valve assembly of FIGS. 1-2 and 4A-4B.

FIG. 5A is an isometric view of a spike of the purge valve assembly of FIGS. 1-2 and 4A-4C.

FIG. 5B is a reverse isometric view of the spike of FIG. 5A.

FIG. 6 is a reverse isometric view of a receiving end portion of the purge valve assembly of FIGS. 1-2 and 4A-4C.

### DETAILED DESCRIPTION

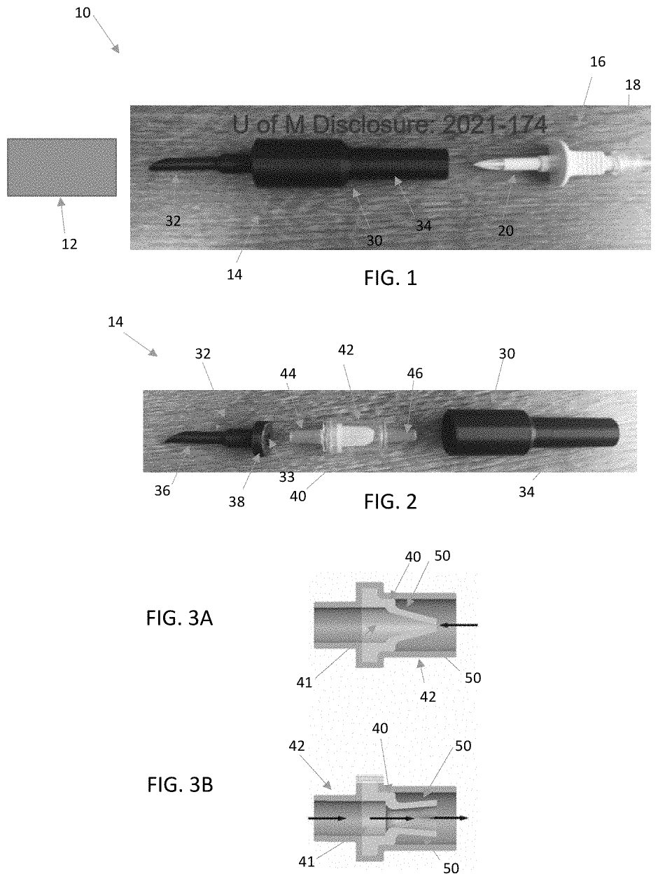

Aspects of the present disclosure relate to systems and methods for preparing the intravenous fluid for infusion, such as rapid infusion. One example of the system is illustrated in FIG. 1. In this example, the system 10 includes a fluid bag 12 (schematically represented) for storing intravenous fluids and potentially containing air, a purge valve assembly 14 and a tubing line assembly 16 including a length of tubing line 18 terminating at a connector 20. In non-limiting examples, the length of tubing can be one of model numbers H-1025 or D-100 available from Smiths Medical ASD, Inc. of Minneapolis, Minn. In one non-limiting example, the connector 20 is a spike as commonly used for puncturing and interconnecting an intravenous fluid bag to tubing, such as the type included with model number H-1025 or D-100 available from Smiths Medical ASD, Inc. of Minneapolis, Minn. In alternate embodiments, the connector 20 can be a Leuer style connection, for example. In one non-limiting example, the fluid bag is a flexible bag made of plastic material or the like. In one example, the fluid bag 12 may have a one-liter storage capacity. When assembled, the purge valve assembly 14 interconnects the fluid bag 12 and the tubing line assembly 16 to create a fluid path from the fluid bag 12 to the tubing 18.

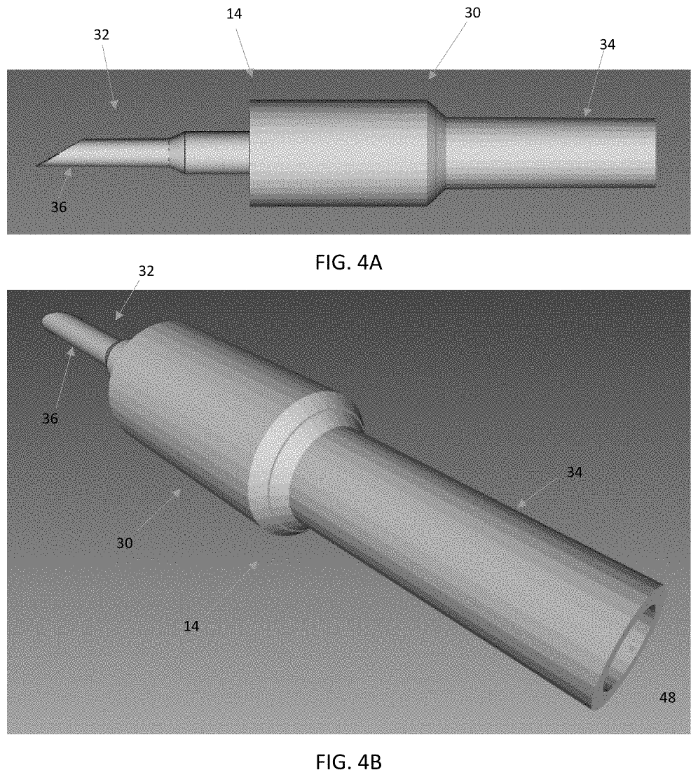

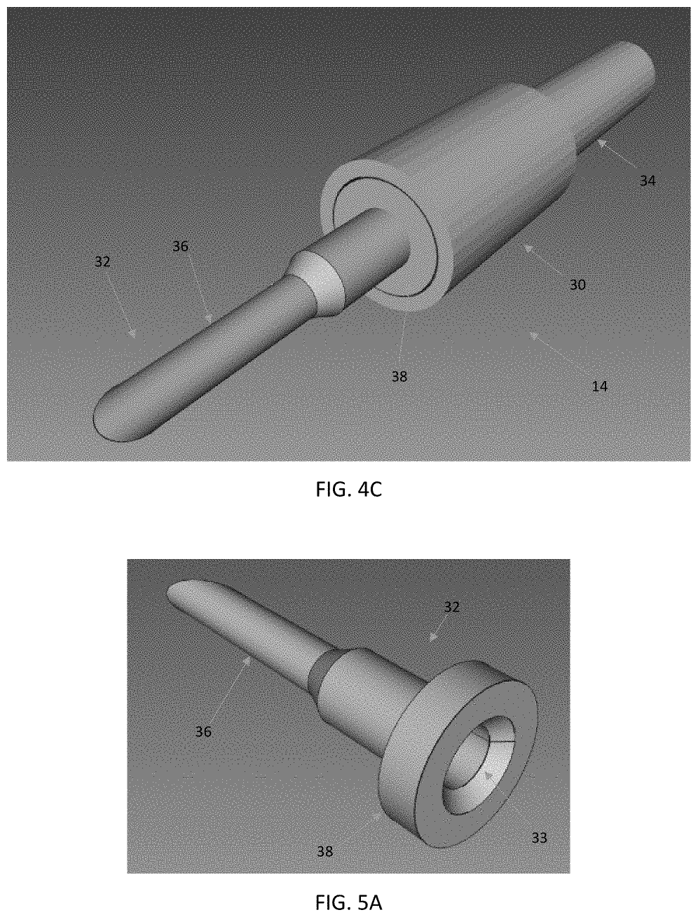

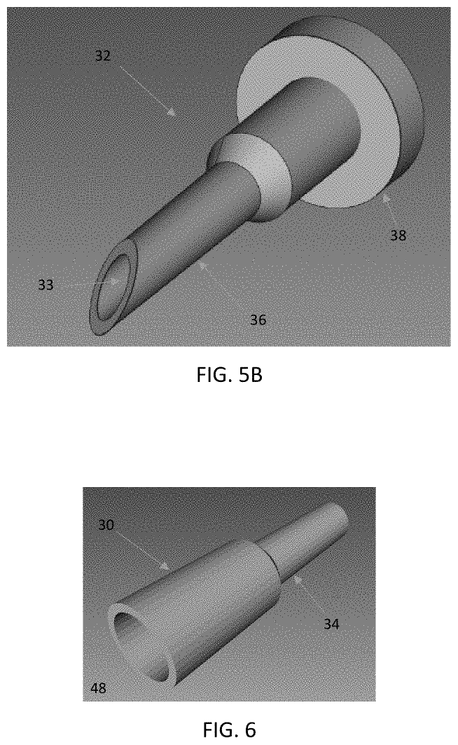

Referring now in addition to FIGS. 2-6, interconnecting the tubing line assembly 16 to the fluid bag 12 is the purge valve assembly 14. The purge valve assembly 14 can include an outer housing 30 connected to a spike 32 for puncturing the fluid bag 12. The outer housing 30 includes a receiving end portion 34 for receiving the connector 20 of the tubing line assembly 16. In some examples, the receiving end portion 34 can include a gasket or other seal to provide a fluid seal between the connector 20 and the receiving end portion 34. In some examples, the spike 32 is similar or identical in configuration to connector 20. Generally, the spike 32 has a channel 33 extending along its length for fluid/air transfer and further includes a tip 36 configured to puncture the fluid bag 12. In this way, the tip 36 can take on a variety of configurations. The spike 32 further includes a base 38 configured to be connected to the outer housing 30. Alternate connections between the spike 32 and outer housing 30 are envisioned. The outer housing 30 additionally houses a check valve 40. Optionally, the check valve 40 can be provided in an inner housing 42. The inner housing 42 may be configured to include a first protrusion 44 that can fit within the channel 33 and a second protrusion 46 hat can fit within the receiving end portion 34. The receiving end portion 34 includes an opening 48 configured to receive and maintain a connection with the connector 20. Once the system 10 is fully assembled, fluid from within the fluid bag 12 can be transferred through the spike 32, through the check valve 40 and then through the receiving end portion 34 and connector 20 to the tubing line 18.

In one example, the check valve 40 is a duckbill style check valve defining a fluid pathway 41 configured to accommodate high flow rates. In one example, a “high” flow rate is a flow rate of at least 25 ml/min but may exceed 1000 ml/min. In this example, the check valve 40 forms conical-like configuration, which allows for self-contained one-way sealing, preventing anything in the tubing line assembly 16 from going past the check valve 40 and into the IV fluid bag 12. The duckbill style check valve 40 also has the advantage at being more robust during manufacturing than other valves since the surface finish quality does not have to be as high. Such a duckbill style check valve 40 can be constructed at least in part from rubber or synthetic elastomer, and includes two or more flaps 50, usually arced and generally shaped like the beak of a duck. The check valve 40 is configured to allow one-way fluid flow through the check valve 40 to prevent contamination due to backflow. A duckbill style check valve is believed to be particularly well suited for the purge valve assemblies of the disclosure because this style of check valve has a relatively low cracking pressure, has a high flow rate with relatively minimal pressure loss, and is less subject to shear forces that could induce hemolysis when infusing blood product. However, membrane style, ball style check valves or the like are also considered to be options for the check valve. In one non-limiting example, the check valve 40 can be model number CV10C available from Pawfly on the Amazon.com marketplace.

Various methods of the disclosure can include the steps of providing the intravenous fluid bag 12 containing fluid and air. Methods can further include providing the tubing line assembly 16 terminating at the connector 20. Methods can further include providing the purge valve assembly 14 having the second spike 32 and the outer housing 30 maintaining the check valve 40. Then, the fluid bag 12 is punctured with the second spike 32. Air is then purged from the fluid bag, through the second spike 32 and out of the check valve 40 and outer housing 30 via opening 48. The outer housing 30 can then be connected to the connector 20. In some methods, the check valve 40 is positioned within the inner housing 42 and the inner housing 42 is further at least partially positioned within the second spike 32. In some methods, the check valve 40 is a duckbill style check valve. In various methods, the second spike 32 includes the tip 36 extending from the base 38, wherein the base is inserted within the outer housing 30. In various methods, after the step of connecting the outer housing 30 to the connector 20, a fluid path is formed from the fluid bag 12, through the purge valve assembly 14 and to the tubing line assembly 16. In some examples, the check valve 40 is a one-way valve restricting fluid from passing from the tubing line assembly 16 to the fluid bag 12 and second spike 32. Optionally, the fluid bag 12 is made of a flexible material. In various methods, the step of purging air from the fluid bag 12 includes flipping the fluid bag over. For example, air can be removed with the purge valve assembly 14 using a technique known as “burping,” which involves turning the fluid bag 12 over before squeezing the air out of the fluid bag. This lowers the amount of air that could possibly get into the tubing line assembly 16 during rapid infusion, lowering the risk of air embolisms.

Various devices and systems of the disclosure eliminate the need for an additional puncture of the fluid bag 12 by the connector 20 by utilizing a method of air extraction from the fluid bag 12 that involves insertion of the purge valve assembly 14. The purge valve assembly 14 allows the user to quickly extract air from the fluid bag 12 without having to remove purge valve assembly 14 from the fluid bag 12. The connector 20 and the tubing line assembly 16 can subsequently be attached to the purge valve assembly 14, completing the setup for rapid infusion, while maintaining a sterile system 10.

Many examples of the disclosure are discussed in relation to aiding in extracting air from an IV fluid bag prior to priming of the tubing line assembly for use in a rapid infuser system. Additionally, it will be understood that aspects of the disclosure can be used in situations where the rapid, or pressurized infusion of IV fluids is necessary including in arterial line transduction and pressurized IV infusion using pneumatic cuffs. Use of the purge valve assemblies of the disclosure in these applications would increase safety of those procedures, as it would prevent introduction of a potentially fatal air embolus. It is further envisioned that another potential application of the purge valve assemblies of the disclosure is use with all infusion devices and are not limited to use with rapid infusion devices. It is envisioned that the purge valve assembly could be used for all IV fluid bags and tubing line assemblies.

Although the present disclosure has been described with reference to preferred embodiments, workers skilled in the art will recognize that changes can be made in form and detail without departing from the spirit and scope of the present disclosure.

What is claimed is:

1. A system comprising:
an intravenous fluid bag;
a tubing line assembly including tubing connected to a connector;
a purge valve assembly including a spike and an outer housing; wherein the outer housing is connected to the connector and includes a receiving end portion configured to receive the connector; wherein the purge valve assembly further includes a check valve provided within the outer housing.

2. The system of claim 1, wherein the check valve is positioned within an interior housing.

3. The system of claim 2, wherein the interior housing is at least partially positioned within the spike.

4. The system of claim 1, wherein the check valve is a duckbill style check valve.

5. The system of claim 1, wherein the spike includes a tip extending from a base, wherein the base can be inserted within the outer housing.

6. The system of claim 1, wherein a fluid path can be formed from the fluid bag, through the purge valve assembly and to the tubing line assembly.

7. The system of claim 1, wherein the check valve is a one-way valve restricting fluid from passing from the tubing line assembly to the fluid bag.

8. The system of claim 1, wherein the fluid bag is made of a flexible material.

9. A method comprising:
providing an intravenous fluid bag containing fluid and air;
providing a tubing line assembly terminating at a connector;
providing a purge valve assembly having a spike and an outer housing maintaining a check valve;
puncturing the fluid bag with the spike;
purging air from the fluid bag, through the spike and out of the check valve; and
connecting the outer housing to the connector.

10. The method of claim 9, wherein the check valve is positioned within an interior housing.

11. The method of claim 10, wherein the interior housing is at least partially positioned within the spike.

12. The method of claim 9, wherein the check valve is a duckbill style check valve.

13. The method of claim 9, wherein the spike includes a tip extending from a base, wherein the base is inserted within the outer housing.

14. The method of claim 9, wherein after the step of connecting the outer housing to the connector, a fluid path is formed from the fluid bag, through the purge valve assembly and to the tubing line assembly.

15. The method of claim 9, wherein the check valve is a one-way valve restricting fluid from passing from the tubing line assembly to the fluid bag.

16. The method of claim 9, wherein the fluid bag is made of a flexible material.

17. The method of claim 9, wherein the step of purging air from the fluid bag includes flipping the fluid bag over.
